(19) United States
(12) Patent Application Publication
(10) Pub. No.: US 2024/0165204 A1
(43) Pub. Date: May 23, 2024

(54) MODULATION OF TUMOR MICROENVIRONMENT TO AUGMENT EFFICACY OF IMMUNOTHERAPY

(71) Applicant: Regen Biopharma, Inc., La Mesa, CA (US)

(72) Inventors: Thomas ICHIM, San Diego, CA (US); Harry LANDER, San Diego, CA (US); David KOOS, La Mesa, CA (US)

(73) Assignee: Regen Biopharma, Inc., La Mesa, CA (US)

(21) Appl. No.: 18/516,884

(22) Filed: Nov. 21, 2023

Related U.S. Application Data
(60) Provisional application No. 63/384,754, filed on Nov. 22, 2022.

(51) Int. Cl. A61K 38/21; A61K 31/166; A61K 31/192; A61P 35/00

(52) U.S. Cl. CPC A61K 38/217; A61K 31/166; A61K 31/192; A61P 35/00

(57) ABSTRACT

Disclosed are compositions of matter and therapeutic means for increasing efficacy of immunotherapy such as Tumor Infiltrating Lymphocytes (TILs), Chimeric Antigen Receptor (CAR)-T cells, and other cellular therapies in solid tumors. In one embodiment agents capable of increasing tumor immunogenicity and/or antigen presentation by modulation of tumor stromal cells such as monocytes and pericytes are administered prior to providing immunotherapy in a patient. In some embodiments administration of interleukin-33 together with one or more antioxidants is provided as a means of stimulating expression of HLA and TAP proteins without inducing significant inflammatory response and myeloid derived suppressor cell activation in cancer associated monocytes. In other embodiments the use of histone deacetylase (HDAC) inhibitors together with interferon alpha is disclosed as a means of stimulating the expression of immunogenic molecules on monocytes to increase immunotherapy cell activity.

### CROSS-REFERENCE TO RELATED APPLICATIONS

This application claims priority to U.S. Provisional Application Ser. No. 63/384,754, titled “Modulation of Tumor Microenvironment to Augment Efficacy of Immunotherapy”, filed Nov. 22, 2022, which is incorporated by reference in its entirety.

### FIELD OF THE INVENTION

The invention relates to immunotherapeutic methods of enhancing cancer associated monocytes for the treatment of cancers.

### BACKGROUND

Cancer therapy has historically been associated with the traditionally terrifying effects of chemotherapy and radiation. Newer approaches have been focused on activities of cancer which are more or less not shared with non-malignant tissue. While chemotherapy and radiation therapy target rapidly proliferating cells, other therapeutic approaches such as angiogenesis inhibition offer a significantly higher degree of selectively in terms of reduced adverse effects. Adult life is more highly dependent on proliferating cells than on angiogenic cells.

Using the immune system to treat cancer is intellectually appealing due to the specificity, memory and efficacy of the immune response in combating traditional pathogens such as bacterial, parasitic or viral infections. Should an anticancer immune response be initiated, the patient could be spared the psychological impact of surgery and/or the toxic side effects of chemotherapy and radiation. The immunotherapy of cancer has been extensively attempted in various forms since the late nineteenth century because of its popular appeal to the patients and the medical community. Interestingly, cancer immunotherapy also has been one of the most controversial fields of science. At the core of the debate regarding whether cancer immunotherapy is of clinical value lies the question, is cancer really a part of nonself? The argument is that since cancer cells are derived from the host, although they are abnormal in certain parameters like increased proliferation or lack of differentiation, they are essentially still a part of the host and cannot be rejected by the immune system. The question thus arises, are there certain immunologically recognizable markers that are specific to cancer cells, and if these markers exist, then, can they be the targets of an immune response? If so, then why is there not a response mounted against cancer in patients that have already succumbed to it? Numerous scientists have investigated these questions for more than a century with opinions swinging on an almost cyclic basis

### SUMMARY

Preferred embodiments are drawn to methods of increasing efficacy of an immunotherapy through augmenting immunogenicity and/or antigen presentation ability of cancer associated monocytes.

Preferred methods include embodiments wherein said cancer associated monocytes produce at least 25% higher levels of interleukin-10 compared to dermal monocytes when cultured under conditions of hypoxia.

Preferred methods include embodiments wherein said hypoxia is 1% oxygen tension.

Preferred methods include embodiments wherein said cancer associated monocytes produce at least 25% higher levels of interleukin-4 compared to dermal monocytes when cultured under conditions of hypoxia.

Preferred methods include embodiments wherein said hypoxia is 1% oxygen tension.

Preferred methods include embodiments wherein said cancer associated monocytes produce at least 50% higher levels of interleukin-13 compared to dermal monocytes when cultured under conditions of hypoxia.

Preferred methods include embodiments wherein said hypoxia is 1% oxygen tension.

Preferred methods include embodiments wherein said cancer associated monocytes produce at least 50% higher levels of interleukin-20 compared to dermal monocytes when cultured under conditions of hypoxia.

Preferred methods include embodiments wherein said hypoxia is 2% oxygen tension.

Preferred methods include embodiments wherein said cancer associated monocytes produce at least 10% higher levels of interleukin-20 compared to dermal monocytes when cultured under conditions of hypoxia.

Preferred methods include embodiments wherein said hypoxia is 2% oxygen tension.

Preferred methods include embodiments wherein said cancer associated monocytes produce at least 10% higher levels of interleukin-35 compared to dermal monocytes when cultured under conditions of hypoxia.

Preferred methods include embodiments wherein said hypoxia is 2% oxygen tension.

Preferred methods include embodiments wherein said cancer associated monocytes produce at least 10% higher levels of TGF-beta compared to dermal monocytes when cultured under conditions of hypoxia.

Preferred methods include embodiments wherein said hypoxia is 2% oxygen tension.

Preferred methods include embodiments wherein said cancer associated monocytes produce at least 30% higher levels of soluble HLA-G compared to dermal monocytes when cultured under conditions of hypoxia.

Preferred methods include embodiments wherein said hypoxia is 2% oxygen tension.

Preferred methods include embodiments wherein said cancer associated monocytes produce at least 50% higher levels of soluble TNF-alpha receptor compared to dermal monocytes when cultured under conditions of hypoxia.

Preferred methods include embodiments wherein said hypoxia is 2% oxygen tension.

Preferred methods include embodiments wherein said cancer associated monocytes produce at least 30% higher levels of interleukin-1 receptor antagonist compared to dermal monocytes when cultured under conditions of hypoxia.

Preferred methods include embodiments wherein said hypoxia is 2% oxygen tension.

Preferred methods include embodiments wherein said cancer associated monocytes produce at least 30% higher levels of VEGF compared to dermal monocytes when cultured under conditions of hypoxia.

Preferred methods include embodiments wherein said hypoxia is 2% oxygen tension.

Preferred methods include embodiments wherein said cancer associated monocytes produce at least 30% higher levels of interleukin-38 compared to dermal monocytes when cultured under conditions of hypoxia.

Preferred methods include embodiments wherein said hypoxia is 2% oxygen tension.

Preferred methods include embodiments wherein said cancer associated monocytes produce at least 30% higher levels of EGF-1 compared to dermal monocytes when cultured under conditions of hypoxia.

Preferred methods include embodiments wherein said hypoxia is 2% oxygen tension.

Preferred methods include embodiments wherein said cancer associated monocytes produce at least 30% higher levels of FGF-1 compared to dermal monocytes when cultured under conditions of hypoxia.

Preferred methods include embodiments wherein said hypoxia is 2% oxygen tension.

Preferred methods include embodiments wherein said cancer associated monocytes produce at least 30% higher levels of FGF-1 compared to dermal monocytes when cultured under conditions of hypoxia.

Preferred methods include embodiments wherein said hypoxia is 2% oxygen tension.

Preferred methods include embodiments wherein said cancer associated monocytes express FAP.

Preferred methods include embodiments wherein said cancer associated monocytes express alpha-SMA/ACTA2.

Preferred methods include embodiments wherein said cancer associated monocytes express MFAP5.

Preferred methods include embodiments wherein said cancer associated monocytes express COL11A1.

Preferred methods include embodiments wherein said cancer associated monocytes express TN-C.

Preferred methods include embodiments wherein said cancer associated monocytes express PDPN.

Preferred methods include embodiments wherein said cancer associated monocytes express ITGA11.

Preferred methods include embodiments wherein said cancer associated monocytes express NG2.

Preferred methods include embodiments wherein said immunogenicity of said cancer associated monocytes is augmented by treatment with interferon gamma and a histone deacetylase inhibitor.

Preferred methods include embodiments wherein said histone deacetylase inhibitor is phenylbutyrate.

Preferred methods include embodiments wherein said histone deacetylase inhibitor is tricostatin A.

Preferred methods include embodiments wherein said histone deacetylase inhibitor is valproic acid.

Preferred methods include embodiments wherein augmentation of immunogenicity is performed by administration of an ERK inhibitor.

Preferred methods include embodiments wherein said ERK inhibitor is administered together with an HDAC inhibitor.

Preferred methods include embodiments wherein said HDAC inhibitor is valproic acid.

Preferred methods include embodiments wherein said HDAC inhibitor is trichostatin A.

Preferred methods include embodiments wherein said HDAC inhibitor is sodium butyrate.

Preferred methods include embodiments wherein inhibition of ERK signaling is achieved by administration of a small molecule ERK inhibitor.

Preferred methods include embodiments wherein said ERK inhibitor is PD0325901.

Preferred methods include embodiments wherein said ERK inhibitor is RDEA119.

Preferred methods include embodiments wherein said ER3K inhibitor is Olomoucine.

Preferred methods include embodiments wherein said ERK inhibitor is Aminopurvalanol A.

Preferred methods include embodiments wherein said ERK inhibitor is AS703026.

Preferred methods include embodiments wherein said ERK inhibitor is AZD8330.

Preferred methods include embodiments wherein said ERK inhibitor is BIX02188.

Preferred methods include embodiments wherein said ERK inhibitor is BIX02189.

Preferred methods include embodiments wherein said ERK inhibitor is CI-1040.

Preferred methods include embodiments wherein said ERK inhibitor is Cobimetirlib.

Preferred methods include embodiments wherein said ERK inhibitor is GDC-0623.

Preferred methods include embodiments wherein said ERK inhibitor is MEk162.

Preferred methods include embodiments wherein said ERK inhibitor is PD318088.

Preferred methods include embodiments wherein said ERK inhibitor is PD98059.

Preferred methods include embodiments wherein said ERK inhibitor is Refametinib.

Preferred methods include embodiments wherein said ERK inhibitor is R04987655.

Preferred methods include embodiments wherein said ERK inhibitor is SCH772984.

Preferred methods include embodiments wherein said ERK inhibitor is Selumetinib.

Preferred methods include embodiments wherein said ERK inhibitor is SL327.

Preferred methods include embodiments wherein said ERK inhibitor is Trametinib.

Preferred methods include embodiments wherein said ERK inhibitor is ARRY-142886.

Preferred methods include embodiments wherein said ERK inhibitor is XL518.

Preferred methods include embodiments wherein increased immunogenicity is achieved by treatment of the patient with dendritic cells.

Preferred methods include embodiments wherein said dendritic cells are obtained from peripheral blood progenitors.

Preferred methods include embodiments wherein said dendritic cells are obtained from mobilized peripheral blood progenitors.

Preferred methods include embodiments wherein said dendritic cells are obtained from menstrual blood progenitors.

Preferred methods include embodiments wherein said dendritic cells are obtained from umbilical cord blood progenitors.

Preferred methods include embodiments wherein said dendritic cells are obtained from monocytic cell progenitors.

Preferred methods include embodiments wherein said dendritic cells are obtained from CD34 progenitors that are treated with GM-CSF and interleukin-4.

Preferred methods include embodiments wherein said dendritic cells are obtained from pluripotent stem cell derived progenitor cells.

Preferred methods include embodiments wherein said pluripotent stem cells are inducible pluripotent stem cells.

Preferred methods include embodiments wherein said dendritic cells are induced to mature after administration in vivo.

Preferred methods include embodiments wherein said maturation is induced by administration of Poly (IC).

Preferred methods include embodiments wherein said maturation is induced by administration of imiquimod.

Preferred methods include embodiments wherein said maturation is induced by administration of HMGB-1.

Preferred methods include embodiments wherein said maturation is induced by administration of CpG motifs.

Preferred methods include embodiments wherein said maturation is induced by administration of xenogeneic cell membranes.

Preferred methods include embodiments wherein said maturation is induced by administration of bacterial cell wall extract.

Preferred methods include embodiments wherein said maturation is induced by administration of beta glucan.

Preferred methods include embodiments wherein said maturation is induced by administration of OK231.

Preferred methods include embodiments wherein said maturation is induced by administration of GM-CSF.

Preferred methods include embodiments wherein said maturation is induced by administration of neutrophil extracellular traps.

Preferred methods include embodiments wherein said maturation is induced by administration of free histones.

Preferred methods include embodiments wherein said maturation is induced by administration of yeast cell wall extract.

Preferred methods include embodiments wherein said maturation is induced by administration of KLH.

Preferred methods include embodiments wherein said maturation is induced by administration of zymosan.

Preferred methods include embodiments wherein said maturation is induced by administration of interferon gamma.

Preferred methods include embodiments wherein said maturation is induced by administration of antibodies to IL-10 or its receptor.

Preferred methods include embodiments wherein said maturation is induced by administration of TNF-alpha.

Preferred methods include embodiments wherein said maturation is induced by administration of IL-33.

Preferred methods include embodiments wherein said maturation is induced by administration of beta defensin.

Preferred methods include embodiments wherein said maturation is induced by administration of complement C3.

Preferred methods include embodiments wherein said maturation is induced by administration of complement C5.

Preferred methods include embodiments wherein said maturation is induced by administration of necrotic cells.

Preferred methods include embodiments wherein augmentation of immunogenicity is achieved by inactivation of said tumor associated monocytes through vaccination against tumor associated monocytes.

Preferred methods include embodiments wherein said tumor monocytes possess an increased ability to efflux rhodamine 123 as compared to monocytes isolated from non-malignant tissues.

Preferred methods include embodiments wherein said vaccine is prophylactic.

Preferred methods include embodiments wherein said vaccine is therapeutic.

Preferred methods include embodiments wherein said vaccine is autologous.

Preferred methods include embodiments wherein said vaccine is allogeneic.

Preferred methods include embodiments wherein said vaccine is xenogeneic.

Preferred methods include embodiments wherein said vaccine is generated from living tumor associated monocytes.

Preferred methods include embodiments wherein said vaccine is generated from mitotically inactivated tumor associated monocytes.

Preferred methods include embodiments wherein said vaccine is generated from tumor associated monocyte necrotic particles.

Preferred methods include embodiments wherein said vaccine is generated from tumor associated monocytes that have undergone the process of pyroptosis.

Preferred methods include embodiments wherein said vaccine is generated from tumor associated monocyte apoptotic bodies.

Preferred methods include embodiments wherein said vaccine is generated from a fusion of tumor associated monocytes and dendritic cells.

Preferred methods include embodiments wherein said tumor associated monocytes fusion with said dendritic cells is accomplished by electroporation means.

Preferred methods include embodiments wherein said tumor associated monocytes fusion with said dendritic cells is accomplished by treatment with polyethelene glycol.

Preferred methods include embodiments wherein said tumor associated monocytes fusion with said dendritic cells is performed using tumor associated monocytes derived from pluripotent stem cells.

Preferred methods include embodiments wherein said pluripotent stem cells are inducible pluripotent stem cells.

Preferred methods include embodiments wherein said pluripotent stem cells are embryonic stem cells.

Preferred methods include embodiments wherein said pluripotent stem cells are somatic nuclear transfer derived stem cells.

Preferred methods include embodiments wherein said pluripotent stem cells are parthenogenic derived stem cells.

Preferred methods include embodiments wherein said tumor associated monocytes fusion with said dendritic cells is performed using tumor associated monocytes derived from hematopoietic stem cells.

Preferred methods include embodiments wherein said hematopoietic stem cells express CD34.

Preferred methods include embodiments wherein said hematopoietic stem cells express CD133.

Preferred methods include embodiments wherein said hematopoietic stem cells express Fas ligand.

Preferred methods include embodiments wherein said hematopoietic stem cells express TRAIL receptor.

Preferred methods include embodiments wherein said hematopoietic stem cells express AIM2.

Preferred methods include embodiments wherein said hematopoietic stem cells express notch.

Preferred methods include embodiments wherein said tumor associated monocytes fusion with said dendritic cells is performed using dendritic cells derived from pluripotent stem cells.

Preferred methods include embodiments wherein said pluripotent stem cells are inducible pluripotent stem cells.

Preferred methods include embodiments wherein said pluripotent stem cells are embryonic stem cells.

Preferred methods include embodiments wherein said pluripotent stem cells are somatic nuclear transfer derived stem cells.

Preferred methods include embodiments wherein said pluripotent stem cells are parthenogenic derived stem cells.

Preferred methods include embodiments wherein said tumor associated monocytes fusion with said dendritic cells is performed using dendritic derived from hematopoietic stem cells.

Preferred methods include embodiments wherein said hematopoietic stem cells express CD34.

Preferred methods include embodiments wherein said hematopoietic stem cells express CD133.

Preferred methods include embodiments wherein said hematopoietic stem cells express Fas ligand.

Preferred methods include embodiments wherein said hematopoietic stem cells express TRAIL receptor.

Preferred methods include embodiments wherein said hematopoietic stem cells express AIM2.

Preferred methods include embodiments wherein said hematopoietic stem cells express notch.

Preferred methods include embodiments wherein said fusion cell is activated prior to administration in a manner capable of increasing immunogenity.

Preferred methods include embodiments wherein said immunogenicity is ability to evoke a recall T cell immune response to one or more cancer associated monocytes induced antigens.

Preferred methods include embodiments wherein said immunogenicity is ability to evoke a recall CD4 T cell immune response to one or more cancer monocyte induced antigens.

Preferred methods include embodiments wherein said immunogenicity is ability to evoke a recall CD8 T cell immune response to one or more cancer monocyte induced antigens.

Preferred methods include embodiments wherein said immunogenicity is ability to evoke a recall NK cell immune response to one or more cancer monocyte induced antigens.

Preferred methods include embodiments wherein said immunogenicity is ability to evoke a recall NKT cell immune response to one or more cancer monocyte induced antigens.

Preferred methods include embodiments wherein said immunogenicity is ability to evoke a recall gamma delta cell immune response to one or more cancer monocyte induced antigens.

Preferred methods include embodiments wherein said immunogenicity is ability to evoke a recall neutrophil immune response to one or more cancer monocyte induced antigens.

Preferred methods include embodiments wherein immunogenicity of said hybrid cell is increased by exposure to agents that increase transporter associated protein expression.

Preferred methods include embodiments wherein said agent that increases transporter associated protein expression is interferon alpha.

Preferred methods include embodiments wherein said agent that increases transporter associated protein expression is interferon gamma.

Preferred methods include embodiments wherein said agent that increases transporter associated protein expression is TNF-alpha.

Preferred methods include embodiments wherein said agent that increases transporter associated protein expression is interleukin-6

Preferred methods include embodiments wherein said agent that increases transporter associated protein expression is interleukin-12.

Preferred methods include embodiments wherein said agent that increases transporter associated protein expression is interleukin-18.

Preferred methods include embodiments wherein immunogenicity of said hybrid cell is increased by exposure to agents that increase MHC I and/or MHC II expression.

Preferred methods include embodiments wherein said agent that increases transporter associated protein expression is interferon alpha.

Preferred methods include embodiments wherein said agent that increases transporter associated protein expression is interferon gamma.

Preferred methods include embodiments wherein said agent that increases transporter associated protein expression is TNF-alpha.

Preferred methods include embodiments wherein said agent that increases transporter associated protein expression is interleukin-6.

Preferred methods include embodiments wherein said agent that increases transporter associated protein expression is interleukin-12.

Preferred methods include embodiments wherein said agent that increases transporter associated protein expression is interleukin-18.

Preferred methods include embodiments wherein immunogenicity of said hybrid cell is increased by exposure to one or more agents capable of triggering the cGAS-STING pathway.

Preferred methods include embodiments wherein immunogenicity of said hybrid cell is increased by exposure to one or more agents capable of triggering the NOD pathway.

Preferred methods include embodiments wherein immunogenicity of said hybrid cell is increased by exposure to one or more agents capable of triggering the toll like receptor (TLR) pathway.

Preferred methods include embodiments wherein one or more immune stimulatory cancer antigens are concurrently administered.

Preferred methods include embodiments wherein said cancer antigens are selected from a group comprising of: ERG, WT1, ALS, BCR-ABL, Ras-mutant, MUC1, ETV6-AML, LMP2, p53 non-mutant, MYC-N, surviving, androgen receptor, RhoC, cyclin B1, EGFRvIII, EphA2, B cell or T cell idiotype, ML-IAP, BORIS, hTERT, PLAC1, HPV E6, HPV E7, OY-TES1, Her2/neu, PAX3, NY-BR-1, p53 mutant, MAGE A3, EpCAM, polysialic Acid, AFP, PAX5, NY-ESO1, sperm protein 17, GD3, Fucosyl GM1, mesothelin, PSMA, GD2, MAGE A1, sLe(x), HMWMAA, CYP1B1, sperm fibrous sheath protein, B7H3, TRP-2, AKAP-4, XAGE 1, CEA, Tn, GloboH, SSX2, RGS5, SART3, gp100, MelanA/MART1, Tyrosinase, GM3 ganglioside, Proteinase 3 (PR1), Page4, STn, Carbonic anhydrase IX, PSCA, Legumain, and MAD-CT-1 (protamin2).

### BRIEF DESCRIPTION OF THE DRAWINGS

FIG. 1 is a bar graph showing the effect of treated cells on increasing expression of HLA II on tumor associated monocyte like-cells.

### DETAILED DESCRIPTION OF THE INVENTION

In one embodiment of the invention, agents are provided which alter the immunological status of the tumor microenvironment in order to induce augmentation of antigen preventing functions. In many tumors cellular components of the tumor microenvironment contribute to localized and in some cases systemic immune suppression. For example, tumor secreted exosomes and/or other microvesicles reprogram macrophages to endow a proangiogenic M2 phenotype. The current invention focuses on altering the tumor microenvironment so as to increase tumor immunogenicity, reduce M2 differentiation, and overall form a microenvironment conducive for successful use of CAR-T cells in solid tumors.

The term “signaling domain” is the functional portion of a protein which acts by transmitting information within the cell to regulate cellular activity via defined signaling pathways by generating second messengers or functioning as effectors by responding to such messengers.

The term “a stromal cell antigen” refers to an antigen expressed on or by a stromal cell. Said stromal cells are tumor associated monocytes, tumor associated monocytes, tumor associated moncytic cells, and tumor associated macrophages.

The term “antigen” or “Ag” as used herein is defined as a molecule that can provoke an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or can be macromolecules besides a polypeptide. Such a biological sample can include, but is not limited to a tissue sample, a tumor sample, a cell or fluid with other biological components.

The term “antigen presenting cell,” as used herein, means an immune system cell such as an accessory cell (e.g., a B-cell, a dendritic cell, and the like) that displays foreign antigens complexed with major histocompatibility complexes (MHC's) on their surfaces. T-cells may recognize these complexes using their T-cell receptors (TCRs). APCs process antigens and present them to T-cells.

The term “anti-tumor effect” as used herein, refers to a biological effect which can be manifested by various means, including but not limited to, a decrease in tumor volume, a decrease in the number of tumor cells, a decrease in the number of metastases, an increase in life expectancy, a decrease in tumor cell proliferation, a decrease in tumor cell survival, or amelioration of various physiological symptoms associated with the cancerous condition. An “anti-tumor effect” can also be manifested by the ability of the peptides, polynucleotides, cells and antibodies of the invention in prevention of the occurrence of tumor in the first place.

The term “auto-antigen” means any self-antigen which is recognized by the immune system as being foreign. Auto-antigens comprise, but are not limited to, cellular proteins, phosphoproteins, cellular surface proteins, cellular lipids, nucleic acids, glycoproteins, including cell surface receptors.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer. The term “combination therapy” refers to a treatment of an individual with at least two different therapeutic agents. According to the invention, the individual is treated with a compound of formula I which constitutes the first therapeutic agent. The second therapeutic agent may be any clinically established anti-cancer therapy, e.g. radiation therapy or administration of a chemotherapeutic drug. A combinatorial treatment may include a third or even further therapeutic agent. In accordance with the invention the combination of HDAC inhibitor and interferon gamma and the third and optionally further therapeutic agent can be administered simultaneously, or the HDAC inhibitor and interferon gamma can be administered prior to or after the their therapeutic agent. Administration of the HDAC inhibitor and interferon gamma prior to the third therapeutic agent or simultaneous administration is preferred. Administration (simultaneously or at a different time) can be done systematically or locally as determined by the indication. In addition, when the third therapeutic agent is radiation therapy, the HDAC inhibitor and interferon gamma can be administered to a cancer patient pre- or post-radiation therapy to treat or ameliorate the effects of cancer. When the first and second and third therapeutic agents are applied at a different time, the time between the three treatments is shorter than 30 days.

The term “histone deacetylase” (HDAC) refers to enzymes that perform the function of removing the acetyl group from lysine residue on histones. To date, 18 mammalian HDACs have been identified and are characterized into four classes: class I HDACs (HDACs 1, 2, 3, and 8), class II HDACs (HDACs 4, 5, 6, 7, 9, and 10), class IV (HDAC 11) and class III (sirtuin family: sirt1-sirt7). Class II HDACs are further divided into two subgroups: class IIa, which has a large C-terminus, and class IIb, which has two deacetylase domains. Class I, II, and IV HDACs need a zinc ion (Zn2+) and share a similar catalytic core for acetyl-lysine hydrolysis, 1 while class III HDACs require a nicotinamide adenine dinucleotide for their enzyme activity.

The term “Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+ T cell) activation, NK cell activation, B cell activation (e.g., antibody generation and/or secretion), binding of an immunogen (e.g., antigen (e.g., immunogenic polypolypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

The term “therapeutically effective amount” refers to the amount of the subject compound that will elicit the biological or medical response of a tissue, system, or subject that is being sought by the researcher, veterinarian, medical doctor or other clinician. The term “therapeutically effective amount” includes that amount of a compound that, when administered, is sufficient to prevent development of, or alleviate to some extent, one or more of the signs or symptoms of the disorder or disease being treated. The therapeutically effective amount will vary depending on the compound, the disease and its severity and the age, weight, etc., of the subject to be treated.

The term “Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells. The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently eintroduced within the context of the current invention. Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemia.

The present invention provides means, therapies and compositions to target the cells of the tumor microenvironment, specifically, stromal cells, more specifically, tumor associated monocytes or cancer associated monocytes as opposed to tumor cells directly, as it was seen that monocytes and fibroblast-like cells existing in the tumor microenvironment have biological activity that impedes efficacy of immunotherapy such as CAR-T cells, or other types of immunotherapy.

For example, stromal cells such as tumor associated monocytes which reside in the tumor microenvironments promote tumor growth and metastasis but also act to reprogram other cells of the immune system to ensure that immunotherapies do not properly function. Therefore, targeting of monocytes to alter their function, to kill them, or to negate their immune suppressive properties will alter in a positive manner the outcome of immunotherapy. In one embodiment Fibroblast Activated Protein expressing monocytes are killed in order to affects a tumor cell so that the tumor cell fails to grow, is prompted to die, or otherwise is affected so that the tumor burden in a patient is diminished or eliminated. In other embodiment administration of agents such as histone deacetylase inhibitors are administered in order to enhance immunogenicity of monocytes that are associated with the cancer cells.

In one embodiment patients suffering from cancer are administered a combination of one or more agents stimulating activation of the interferon gamma pathway together with one or more histone deacetylase inhibitors. One of the findings disclosed in the current invention is that the combination of interferon gamma and valproic acid synergistically increases expression of HLA II on monocytes (Example 1). HLA II is needed for monocytes to activate immunity and is a marker of tumor associated monocytes changing immunological propensity from tolerogenicity to immunogenicity.

In one embodiment of the invention, immune stimulants that induce the production of interferon gamma are utilized together with histone deacetylase inhibitors in order to generate monocytes that promote immunity and allow for function of CAR-T cells as opposed to inhibitor monocytes.

In one embodiment immune stimulants capable of inducing the body to produce interferon gamma are isolated Staphylococcal enterotoxin. It is known that staphylococcal enterotoxins A, B, C, D, E, toxic shock toxin (TSST-1), a product of Mycoplasma arthritidis, mycobacterial species, heat shock peptides and Mls antigens provoke dramatic T cell responses. Staphylococcal enterotoxins are the most powerful T cell mitogens known eliciting strong polyclonal proliferation at concentrations even a 1000 times lower than such conventional T cell mitogens as phytohemagglutinin. The administration of these proteins can be performed prior to, and/or concurrent with, and/or subsequently to administration of one or more histone deacetylase inhibitors. For the practice of the invention, SEA can be utilized in some embodiments since it is the most potent T cell mitogen, stimulating DNA synthesis at concentrations of 10-13 to 10-16 M in the human system. All stimulate a large proportion of both murine and human CD4+ and CD8+ T cells. Activity of these mitogens is tightly restricted by the major histocompatibility complex (MHC) class II antigens. It is proposed that the staphylococcal enterotoxins, streptococcal pyrogenic exotoxins, exfoliative toxins and a product of mycoplasma arthritis bind directly to the T cell receptor and to class II MHC. These two structures are brought into contact, thus stimulating T cell activation via the V3 region of the T cell receptor mimicking strong alloreactive response. Various types of Enterotoxins may be utilized for the practice of the invention. For example, Enterotoxin B, which can be isolated from Staphylococcus aureus, that is used for the production of SEB (Staphylococcal enterotoxin B) is e.g., S6 or 10-275. In one embodiment, the medium containing the toxin is diluted twice with water adjusted to a pH of 6.4, and AmberLite CG-50 (200 mesh) cation ion-exchange resin is added to the toxin mixture. The toxin is eluted, dialyzed, then reapplied to the CG-50 column again. The eluted toxin is dialyzed, then applied to a column of carboxymethyl cellulose or CM-Sephadex. Unbound proteins are eluted with 0.03 and 0.04 molar sodium phosphate buffer. At this point, the toxin is essentially homogeneous. Using chromatofocusing techniques, the SEB may be further subdivided into several isoelectric species using polybuffer 96. Enterotoxin A (SEA) can be obtained form high SEA producers, e.g., Staphylococcus aureus 13M-2909 are grown in the general medium that is made 0.2% in glucose. Initially, AmberLite CG-50 is used for batch isolation. After incubation, the toxin is eluted and dialyzed. The toxin is then loaded onto a CM-cellulose column and eluted with a linear gradient. The combined fractions are then loaded onto a hydroxylapatite column and eluted using a linear gradient. The fractions are lyophilized and chromatographed on a Sephadex-G-75 column. The toxins obtained from this procedure are greater than 99% pure, with a yield of approximately 20%. Eterotoxin C1 (SEC1) can be purified from culture supernatant of Staphylococcus aureus 137 which is concentrated, dialyzed and lyophilized. The toxin product is then applied to a carboxymethyl cellulose column and eluted with a stepwise gradient. The toxin peak consists of a sharp peak with a trailing edge. The eluted toxin is concentrated and applied to Sephadex-G-75. The toxin elutes as a single peak. The toxin is then concentrated and run twice through a column of Sephadex-G-50. The eluate is dialyzed against water and lyophilized. Enterotoxin C2 (SEC2) can be isolated from the culture supernatant from Staphylococcus aureus 361 which is concentrated as for SEC1 and dialyzed. The toxin is then applied to a carboxymethyl cellulose column. SEC2 is eluted, lyophilized and resuspended in distilled water. The toxin is reapplied to a column of carboxymethyl cellulose and eluted with a linear gradient. The partially purified toxin is concentrated and applied to a Sephadex-G-75 column. The eluted toxin is concentrated and finally reapplied to a Sephadex-G-50 column. Recovery is about 40%, with purity exceeding 99%. Enterotoxin D (SED) can be purified from Staphylococcus aureus 1151M which is used for the production of enterotoxin B. The medium is similar to that used for SEA and SEB. After growth and removal of the cells, the pH of the supernatant is adjusted to 5.6 and applied to an AmberLite-CG-50 resin. The mixture is stirred for one hour, and the toxin is eluted and concentrated using 20% (W/V) polyethylene glycol, 20M. The concentrated toxin is dialyzed and applied to a carboxymethyl cellulose column. The toxin is eluted in a linear gradient and then rechromatographed on carboxymethyl cellulose. The toxin solution is concentrated and chromatographed on Sephadex-G-75. This step is repeated once. Enterotoxin E (SEE) can be purified from Staphylococcus aureus strain PRI-236 culture supernatant is concentrated and dialyzed. The toxin is then absorbed to a carboxymethyl cellulose column. The toxin is eluted in a stepwise fashion and concentrated. It is then chromatographed twice on Sephadex-G-75. To obtain highly purified SEE, it is necessary to chromatograph the toxin once more on G-75 in the presence of 6 molar urea. Enterotoxin F or Toxic Shock Syndrome Toxin-1 (TSST-1), TSST-1a and TSST-1b can be purified from the Staphylococcus strain MN8 which is cultured overnight in dialyzable beef heart medium and precipitated from culture fluid by adding 4 volumes of absolute ethanol and storing for at least 2 days. The precipitate is collected by centrifugation and the pellet is suspended in water, recentrifuged and dialyzed to remove salts. The preparation is then electrofocused in a pH gradient of 3-10 using commercial ampholytes with the LKB Multiphor apparatus. The visible band containing TSST-1 is harvested and refocused in a pH 6-8 gradient yielding purified TSST-1. TSST-1a and 1b are isolated by one additional electrofocusing step. After focusing TSST-1 on the pH 6-8 gradient, approximately one-half of the Sephadex gel is removed from the anode end. The gel remaining on the cathode end, containing the TSST-1 band is repoured after the addition of two more grams of Sephadex gel and then refocused overnight using the remaining pH gradient. After electrofocusing in a pH 6-8 or 6.5-7.5 gradient, protein bands are located by the zymogen print method. Discrete bands are scraped off the plate and eluted with pyrogen free water from the Sephadex gel. Strain MN8 yields approximately 2 mg of each toxin per liter of culture fluid. For Staphylococcus aureus strains other than MN8, 200 μg of each toxin is obtained per liter of culture fluid. TSST-1a and 1b are proteins which migrate as homogeneous bands in SDS gels to a molecular weight of 22,000 with isoelectric points of 7.08 and 7.22, respectively.

In some embodiments of the invention HDAC inhibitors such as valproic acid are added together with antigen presenting cells. In some embodiments dendritic cells are used to generate T cells that in vivo trigger production of interferon gamma. In some embodiments tumor lysates may be added into said patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro and rendering them resistant to tumor derived inhibitory compounds such as arginase byproducts. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to said T cells and/or NK cells in order to endow cytotoxic activity towards cancer and/or cancer associated monocytes cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. The immune response of the patient treated with these cytotoxic cells is assessed utilizing a variety of antigens found in tumor cells. When cytotoxic or antibody, or antibody associated with complement fixation are recognized to be upregulated in the cancer patient, subsequent immunizations are performed utilizing peptides to induce a focusing of the immune response.

In another embodiment DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a PlY kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generates a peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/mL GM-CSF and 30 ng/mL IL-4. On day 6 immature dendritic cells are pulsed with tumor antigen. Pulsing may be performed by incubation of lysates with dendritic cells, or may be generated by fusion of immature dendritic cells with tumor cells. Means of generating hybridomas or cellular fusion products are known in the art and include electrical pulse mediated fusion, or stimulation of cellular fusion by treatment with polyethelyne glycol. On day 7, the immature DCs are then induced to differentiate into mature DCs by culturing for 48 hours with 30 ng/mL interferon gamma (IFN-7). During the course of generating DC for clinical purposes, microbiologic monitoring tests are performed at the beginning of the culture, on the fifth day and at the time of cell freezing for further use or prior to release of the dendritic cells. Administration of tumor pulsed dendritic cells is utilized as a polyvalent vaccine, whereas subsequent to administration antibody or t cell responses are assessed for induction of antigen specificity, peptides corresponding to immune response stimulated are used for further immunization to focus the immune response.

In some embodiments, culture of the immune effectors cells is performed after extracting from a patient that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD40L, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects. Said cells can be expanded in the presence of specific antigens associated with tumors and subsequently injected into the patient in need of treatment. Expansion with specific antigens includes coculture with proteins selected from a group comprising of: a) ROBO; b) VEGF-R2; c) FGF-R; d) CD105; e) TEM-1; and f) survivin. Other tumor specific or semi-specific antigens are known in the art that may be used.

In some embodiments of the invention addition of a neutrophil elastase inhibitor is utilized together with the interferon gamma inducing enterotoxin together with the histone deacetylase inhibitor. One useful neutrophil elastase inhibitor is alpha 1 antitrypsin. In some embodiments alpha 1 antitrypsin is administered prior to, and/or concurrently with, and/or subsequent to interferon gamma and/or valproic acid in order to suppress downregulation of MHC II by neutrophil elastase mediated cleavage. In order to better understand the invention, and to assist in its practice, we will describe the function and role of histone deacetylases. In mammals the histone deacetylases can be divided into three subclasses: a) Class I enzymes are homologues of the yeast RPD3 protein and include the mammalian HDAC1, HDAC2, HDAC3 and HDAC8 enzymes with molecular masses ranging from 42 to 55 kDa; b) Class II histone deacetylases HDAC4, HDAC5, HDAC6 and HDAC7 are larger proteins (about 120 to 130 kDa) which are related to the yeast HDA1 protein; and c) Third class of histone deacetylases with homology to the yeast SIR2 protein and several putative mammalian members has been identified.

It is recognized, scientifically, that the enzymes known as histone deacetylases bind to many different proteins and usually exist in large complexes within the cell. Many of the associated proteins seem to be involved in either targeting HDACs to their substrates or to transcriptional repressors. For example, the Rb-associated proteins RbAP46 and RbAP48 are usually considered as integral parts of the HDAC enzyme complex responsible for the recognition of nucleosomal targets. The corepressors N—CoR, SMRT and Sin3 on the other hand are bridging factors required for the recruitment of HDACs to transcription factors. Histone deacetylases are also components of the nucleosome remodeling and deacetylase (NuRD) complex which also contains RbAP46 and RbAP48, Mi-2 and MTA2. Given the large number of HDAC isoenzymes and interacting proteins it is conceivable that complex composition could modulate substrate specificity and target HDACs even to non-histone proteins.

Within the current invention and described the above methods for cancer treatment may be combined together and/or may be combined with other known methods for treating a particular cancer. The concept that the invention is disclosing is alteration of the tumor microenvironment, specifically of the barriers to immunological entry into the tumor. Through breaking this barrier many other therapies can benefit. Treatments of cancer may include such methods such as chemotherapy, surgery, radiotherapy, photodynamic therapy, gene therapy, antisense therapy, enzyme prodrug therapy, immunotherapy, fusion toxin therapy, antiangiogenic therapy, or any combination of these therapies. In this embodiment, preferably, the histone deacetylase inhibitor is administered systemically or it may be delivered locally. In some embodiments, hexosaminidase may be added either chitinase, chitosanase, or N-acetyl-hexosaminidase, and the targeting ligand is alternately a monoclonal antibody or antibody fragment immunospecific to a tumor cell or cancer cell antigen, epidermal growth factor (EGF), fibroblast growth factor (FGF), transferrin, folic acid, or any other molecule that selectively binds to the tumor stroma or even to the cancer cell. The histone deacetylase inhibitors, as well as the stimulators of interferon gamma signaling of the present invention can be administered to humans in an amount that inhibits the immune suppression caused by the tumor.

Quantification of immune suppression may be performed through various means known in the art. In some embodiments quantification of tumor specific T cells is be performed by use of tetramer technologies or tetramer analysis. This is a process that uses special proteins on the surface of cells, called major histocompatibility complexes (MHCs), to detect T-lymphocytes, or T-cells, to determine whether or not a person has a disease. T-cells are a type of immune cell that go on search and destroy missions for diseased and cancerous cells. If they find one, they destroy it. If a T-cell is in a patient's body, it means they have the disease because their body is trying to fight it. Tetramer analysis will find the T-cell, and quantify the number of T cells. In some embodiments the T cell is assessed for activation status by quantitative and/or qualitative staining. MHC class II monomers are produced in a mammalian expression system by linking the peptide of interest to the gene of N-terminal of 3-chain and co-transfecting with α-chain. All MHC class I monomers produced by the IML are folded using a photocleavable ligand. Upon exposure to UV light, the photocleavable ligand is broken thereby allowing your peptide of interest to be exchanged into the binding site of the MHC class I monomer. The peptide-exchanged class I monomer and class II monomer are then converted to tetramers by labeling with streptavidin conjugated fluorochrome and used for cell staining. The use of combinatorial coding of tetramers allows for high throughput parallel detection of antigen-specific T cells and also greatly decreases non-specific background within samples thereby allowing visualization of small tetramer-positive populations within a single sample.

In some embodiments of the invention interferon and valproic acid will be administered together with chemotherapy and immunotherapy. In some embodiments various types of chemotherapy will be used. Known chemotherapies include but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

In one embodiment, leukocytes are taken from the subject's body using leukapheresis. T cells are enriched and washed to separate them from the leukocytes. T cell subsets at the level of CD4/CD8 composition are separated using specific antibody bead conjugates or markers. Cultures are then established to activate the T cells using one or more of the following: autologous antigen-presenting cells (APCs) purified from the subject, beads coated with anti-CD3/anti-CD28 monoclonal antibodies, or anti-CD3 monoclonal antibodies alone or in combination with feeder cells or growth factors. IL-2 is an example of a growth factor that may be used in this context. Subsequently, CARs are then introduced into the T cells using methods known in the art, for example, using lentiviral vectors, gammaretroviral vectors, the Sleeping Beauty transposon system, mRNA transfection, or other methods. Bioreactor systems are used to culture the CAR T cells, for example, WAVE Bioreactor, G-Rex, CliniMACS Prodigy or TERUMO Quantum system. Static culture bags or perfusion systems that allow controlled dynamic medium and gas exchange may be used for the cultivation of CAR cells. Other cell cultivation platforms known in the art that are used to manufacture large quantities of individual immune cells may also be employed in the context of the invention. Prior to, and/or concurrent with, and/or subsequent to administration of CAR-T, a combination of interferon gamma and histone deacetylase inhibitor is administered such as valproic acid. Assessment of immunogenicity and/or success of the therapy can be performed by assessment of HLA II expression on tumor derived monocytes. This may be assessed by tumor biopsy or it may be assessed by utilization of monocyte exosomes as a means of quantification. In some embodiments quantification of efficacy is assessed by growth of a tumor or, preferably, eliminates the tumor from the body. In other embodiments the approach will be varied since the tumor will vary with the particular tumor being treated, the age and physical condition of the subject being treated, the severity of the condition, the duration of the treatment, the nature of the concurrent therapy (if any), the specific route of administration and like factors within the knowledge and expertise of the health practitioner. In the event that a response in a subject is insufficient at the initial doses applied, higher doses (or effectively higher doses by a different, more localized delivery route) may be employed to the extent that patient tolerance permits. Multiple doses are contemplated to achieve good results.

### Example

Increasing Expression of HLA II on Tumor Associated Monocyte Like-Cells

Peripheral blood derived monocytes are obtained from ATCC and cultured in complete DMEM media containing 100 ng/ml PGE2 and 20 ng/ml TGF-beta to mimic tumor associated monocytes. Cultures are performed for 7 days with media changes every 3 days. Cells are passaged with trypsin obtained from ThermoFisher.

Cells are treated with control media, Interferon Gamma (10 ng/ml), Valproic Acid (100 ng/ml) or the combination. Cells are collected and assessed for expression of HLA II by flow cytometry at the indicated timepoints.

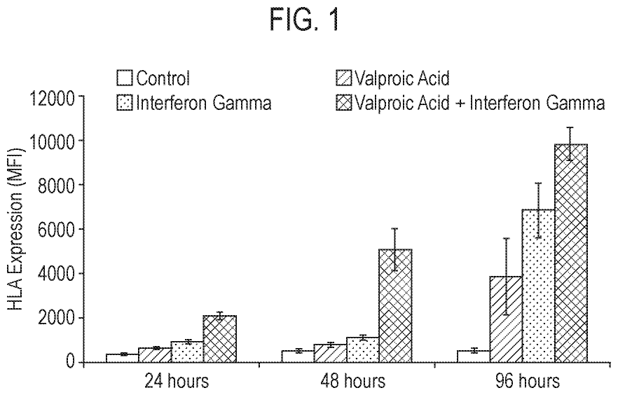

A synergistic increase in HLA II is observed indicating augmented immunogenicity of the treated cancer associated monocytes. Results are shown in FIG. 1

Raw Data

| | 24 Hours | Valproic | Interferon | Valproic Acid + |
|---|---|---|---|---|
| | Control | Acid | Gamma | Interferon Gamma |
| 1.0 | 323.0 | 435.0 | 876.0 | 2033.0 |
| 2.0 | 334.0 | 664.0 | 997.0 | 2003.0 |
| 3.0 | 364.0 | 546.0 | 975.0 | 1998.0 |
| 4.0 | 347.0 | 547.0 | 896.0 | 1976.0 |
| 5.0 | 443.0 | 754.0 | 888.0 | 1984.0 |
| 6.0 | 436.0 | 678.0 | 807.0 | 1988.0 |
| 7.0 | 334.0 | 764.0 | 905.0 | 2005.0 |
| 8.0 | 375.0 | 568.0 | 956.0 | 2124.0 |
| 9.0 | 312.0 | 658.0 | 896.0 | 2245.0 |
| 10.0 | 254.0 | 646.0 | 985.0 | 2535.0 |
| Average | 352.2 | 626.0 | 918.1 | 2089.1 |
| STDEV | 56.5 | 101.9 | 59.2 | 177.7 |

| | 48 Hours | Valproic | Interferon | Valproic Acid + |
|---|---|---|---|---|
| | Control | Acid | Alpha | Interferon Alpha |
| 1.0 | 435.0 | 878.0 | 1002.0 | 4367.0 |
| 2.0 | 467.0 | 768.0 | 1124.0 | 4463.0 |
| 3.0 | 545.0 | 875.0 | 1263.0 | 4347.0 |
| 4.0 | 557.0 | 674.0 | 1007.0 | 4367.0 |
| 5.0 | 573.0 | 576.0 | 997.0 | 4347.0 |
| 6.0 | 537.0 | 787.0 | 1125.0 | 4578.0 |
| 7.0 | 653.0 | 769.0 | 1072.0 | 5634.0 |
| 8.0 | 347.0 | 884.0 | 1125.0 | 6524.0 |
| 9.0 | 547.0 | 769.0 | 1363.0 | 6657.0 |
| 10.0 | 523.0 | 880.0 | 1126.0 | 5458.0 |
| Average | 518.4 | 786.0 | 1120.4 | 5074.2 |
| STDEV | 84.0 | 101.3 | 117.2 | 927.7 |
| | 96 Hours | Valproic | Interferon | Valproic Acid + |
| | Control | Acid | Alpha | Interferon Alpha |
| 1.0 | 456.0 | 2976.0 | 4575.0 | 10425.0 |
| 2.0 | 543.0 | 2235.0 | 5547.0 | 9979.0 |
| 3.0 | 543.0 | 3231.0 | 5769.0 | 9086.0 |
| 4.0 | 765.0 | 3364.0 | 7655.0 | 8868.0 |
| 5.0 | 436.0 | 3467.0 | 7548.0 | 9066.0 |
| 6.0 | 533.0 | 4347.0 | 7684.0 | 10242.0 |
| 7.0 | 554.0 | 4342.0 | 8876.0 | 11264.0 |
| 8.0 | 346.0 | 4675.0 | 9856.0 | 10036.0 |
| 9.0 | 467.0 | 6653.0 | 5457.0 | 9678.0 |
| 10.0 | 564.0 | 3246.0 | 5532.0 | 9784.0 |
| Average | 520.7 | 3853.6 | 6849.9 | 9842.8 |
| STDEV | 109.8 | 1226.1 | 1722.1 | 725.0 |

1. A method of increasing efficacy of an immunotherapy through augmenting immunogenicity and/or antigen presentation ability of cancer associated monocytes.

2. The method of claim 1, wherein said cancer associated monocytes produce at least 25% higher levels of interleukin-10 compared to dermal monocytes when cultured under conditions of hypoxia.

3. The method of claim 2, wherein said hypoxia is 1% oxygen tension.

4. The method of claim 1, wherein said cancer associated monocytes produce at least 25% higher levels of interleukin-4 compared to dermal monocytes when cultured under conditions of hypoxia.

5. The method of claim 1, wherein said cancer associated monocytes produce at least 30% higher levels of FGF-1 compared to dermal monocytes when cultured under conditions of hypoxia.

6. The method of claim 5, wherein said hypoxia is 2% oxygen tension.

7. The method of claim 1, wherein said cancer associated monocytes express FAP.

8. The method of claim 1, wherein said cancer associated monocytes express alpha-SMA/ACTA2.

9. The method of claim 1, wherein said cancer associated monocytes express MFAP5.

10. The method of claim 1, wherein said immunogenicity of said cancer associated monocytes is augmented by treatment with interferon gamma and a histone deacetylase inhibitor.

11. The method of claim 10, wherein said histone deacetylase inhibitor is phenylbutyrate.

12. The method of claim 10, wherein said histone deacetylase inhibitor is tricostatin A.

13. The method of claim 10, wherein said histone deacetylase inhibitor is valproic acid.

14. The method of claim 1, wherein augmentation of immunogenicity is performed by administration of an ERK inhibitor.

15. The method of claim 14, wherein said ERK inhibitor is administered together with an HDAC inhibitor.

16. The method of claim 15, wherein said HDAC inhibitor is valproic acid.

17. The method of claim 15, wherein said HDAC inhibitor is trichostatin A.

18. The method of claim 15, wherein said HDAC inhibitor is sodium butyrate.

19. The method of claim 14, wherein inhibition of ERK signaling is achieved by administration of a small molecule ERK inhibitor.

20. The method of claim 19, wherein said ERK inhibitor is PD0325901.
